(19) United States
(12) Patent Application Publication
(10) Pub. No.: US 2026/0193711 A1
(43) Pub. Date: Jul. 9, 2026

(54) METHOD OF PREDICTION OF PREGNANCY COMPLICATIONS ASSOCIATED WITH A HIGH RISK OF PREGNANCY LOSS BASED ON THE EXPRESSION PROFILE OF CARDIOVASCULAR MiRNAs

(71) Applicant: GENESPECTOR S.R.O., Praha (CZ)

(72) Inventor: Ilona HROMADNIKOVA, Praha (CZ)

(21) Appl. No.: 19/134,730

(22) PCT Filed: Aug. 8, 2023
(86) PCT No.: PCT/CZ2023/050049

§ 371 Date: Jun. 1, 2025

(30) Foreign Application Priority Data
Dec. 2, 2022 (CZ) PV 2022-505

(51) Int. Cl. C12Q 1/6883

(52) U.S. Cl. CPC C12Q 1/6883; C12Q 2600/118; C12Q 2600/158; C12Q 2600/178; G01N 2800/368

(57) ABSTRACT

Method of prediction of pregnancy complications associated with a high risk of pregnancy loss, such as miscarriage, stillbirth, or HELLP syndrome. Pregnant women are screened to determine the expression profile of two or more miRNAs in whole peripheral venous blood collected in the period of 10th-13th gestational week, whereas said two or more miRNAs are selected from the group miR-1-3p, miR-16-5p, miR-17-5p, miR-20a-5p, miR-26a-5p, miR-130b-3p, miR-143-3p, miR-145-5p, miR-146a-5p, miR-181a-5p, miR-195-5p, miR-210-3p, miR-342-3p, miR-499a-5p a miR-574-3p.

### TECHNICAL FIELD

The invention relates to the field of analysis of non-coding nucleic acids and expression markers, specifically microRNAs (miRNAs), using molecular-biological methods, primarily quantitative polymerase chain reaction with reverse transcription (RT-qPCR), and their use in screening to predict the development of critical complications in pregnancy.

### BACKGROUND ART

During pregnancy, the mother or the fetus can experience several complications that significantly contribute to maternal and perinatal morbidity and mortality, with complications associated with pregnancy loss being the most serious from both social and clinical point of view. Pregnancy loss is generally defined as the death of an unborn child at any time during pregnancy. Miscarriage is defined as the spontaneous termination of pregnancy when an embryo or fetus showing no signs of life is expelled or removed from the uterus and its weight is less than 500 g. If it is not possible to determine the weight, the determining criterion is whether the pregnancy in question was shorter than 22 weeks. A stillbirth means a fetus born without signs of life weighing 500 g or more. If it is not possible to determine the birth weight, the determining criterion is whether the baby was born after the 22nd completed week of pregnancy, or, in case the length of the pregnancy cannot be determined, whether the child was at least 25 cm long, from the top of the head to the heel. A life-threatening pregnancy complication characterized by a very high—up to 40%—perinatal and maternal mortality, i.e., death of the child or the mother, is the so-called HELLP syndrome (Hemolysis, Elevated Liver enzymes, Low Platelets). While the pathogenesis is unclear, it is probably a generalized vasospasm, i.e., a spasmodic narrowing of blood vessels, with a subsequent syndrome of multiorgan failure, especially of the liver and kidneys, and an activation of coagulation.

The above-mentioned pregnancy complications significantly affect both mental and physical health of mothers. Above all, HELLP syndrome is associated with significantly higher maternal and neonatal mortality and morbidity. Therefore, a follow-up monitoring and professional counseling are very important for the affected women. As a result, these complications, if not detected in time, also represent a significant burden on the healthcare system. Thus, the aim of contemporary medicine is to predict these complications in time and, ideally, to prevent them from occurring or at least to mitigate their course and effects through early intervention. However, in current clinical practice, there are no ways to predict complications such as miscarriage, stillbirth, or HELLP syndrome. It is especially difficult in first-time mothers, who do not have a medical history that a doctor could use as a basis.

Documents Hromadnikova et al., Biomedicines 2022, 10, p 256; Hromadnikova et al., Biomedicines 2022, 10, p 718; Hromadnikova et al., Int. J. Mol. Sci. 2022, 23, p 3951; Hromadnikova et al., Int. J. Mol. Sci. 2022, 23, p 10635 describe methods for predicting pregnancy complications in the form of preeclampsia, fetal growth restriction, gestational hypertension, small fetal size for a given gestational age, preterm birth (in the form of spontaneous preterm birth or preterm premature rupture of membranes), and gestational diabetes mellitus, as well as detection of undiagnosed chronic hypertension, using selected cardiovascular miRNAs as biomarkers. However, since the physiological causes of complications associated with pregnancy loss are, albeit known, extremely variable and HELLP syndrome—the causes of which are on the contrary not clearly known—is characterized by a multiorgan failure, it is not possible to use the knowledge from the cited documents, i.e., the use of cardiovascular miRNAs, in an obvious way for the development of a method for predicting miscarriage, stillbirth, or HELLP syndrome.

Examples of changes in levels of some miRNAs associated with death of the fetus during pregnancy have been described in scientific literature. Document Vahid et al., J. Cell. Physiol. 2019, 234, p 4924-4933 describes changes in levels of miRNA associated with the immune system occurring in activated peripheral blood mononuclear cells in women with recurrent pregnancy loss (RPL), i.e., women who have had at least two miscarriages before 20th week of pregnancy. However, the cited study does not indicate when the miRNA levels were analyzed, i.e., whether during or after pregnancy. Document Jairajpuri et al., Gene 2021, 768, p 145334 describes an altered expression profile of some circulating plasma miRNAs in women suffering from an early RPL characterized by miscarriages during the 8th-12th week of pregnancy and only mentions—but does not prove—the possibility of using these findings to predict the onset of an early RPL. In addition, the cited document describes miRNAs circulating freely in plasma or as a part of plasma exosomes. The processing of plasma exosomes is a demanding process that is not suitable for wider use in clinical practice. Document Ding et al., Theranostics 2021, 11(12), p 5813 describes an in vitro study, where miR-146a-5p and miR-146b-5p in extracellular vesicles derived from M1 macrophages from the THP-1 cell line were observed to suppress migration and invasion of placental-tissue trophoblasts in a recurrent spontaneous miscarriage. However, the document does not discuss the possibility of using these miRNAs to predict miscarriage or any other pregnancy complications. Moreover, findings from these three documents cannot be applied to women without a previous history of pregnancy.

Goal of the present invention is to eliminate the drawbacks of the prior art by developing a method that can be easily implemented on a large scale even in a commonly equipped molecular-genetics laboratory and which is able to predict—already during the first trimester of pregnancy—in a robust manner and with high reliability miscarriage, stillbirth, or HELLP syndrome, i.e., pregnancy complications for which there is no possibility of prediction in current clinical practice, and to do so even in first-time mothers or with anonymous samples, i.e., without the need to know the data about the mother and her clinical parameters.

### SUMMARY OF INVENTION

The present invention is based on determining levels of 15 specific miRNAs (miR-1-3p, miR-16-5p, miR-17-5p, miR-20a-5p, miR-26a-5p, miR-130b-3p, miR-143-3p, miR-145-5p, miR-146a-5p, miR-181 a-5p, miR-195-5p, miR-210-3p, miR-342-3p, miR-499a-5p, miR-574-3p) by an RT-qPCR method in samples of whole peripheral venous blood taken from pregnant women during the standard first-trimester screening, i.e., in the period of 10th to 13th gestational week. This way, it is possible to predict—with high probability and reliability—the onset of pregnancy complications associated with a high risk of pregnancy loss. By selecting suitable subsets from this group of miRNAs, it is possible to specifically predict miscarriage, stillbirth, and HELLP syndrome, respectively.

Although the connection between changes in levels of the above-mentioned miRNAs and the prediction of pregnancy complications associated with the abnormal function of the placenta and the mother's cardiovascular system has already been presented in scientific documents, and is therefore known from the prior art, their use for the prediction of miscarriage, stillbirth, or HELLP syndrome, i.e., pregnancy complications associated with a high risk of pregnancy loss, is not obvious or easily deducible from these documents. The reason being that complications associated with pregnancy loss have a number of very different causes (e.g., genetic diseases and disorders, imbalance of sex hormones, disorders of the immune system or endocrine glands, infectious and hematological diseases, congenital defects and anomalies of the uterus, older age of mothers, or unhealthy lifestyle). Another reason is the large number of described miRNAs, their specific connection with the cardiovascular system, the high variability of possible changes in expression levels, and the necessity of non-obvious selection of a specific subset of 15 of these markers.

The method was developed based on analyses of selected samples from 12,000 women in the first trimester of pregnancy, statistical processing of the results of these analyses, and their comparison with the subsequent course of pregnancy in the monitored women.

The method according to the invention is carried out as follows. First, the collected blood is processed into a leukocyte lysate. The contained RNA is then extracted and short RNAs are subsequently isolated. Resulting purified solution is analyzed using a two-step RT-qPCR reaction in a device maintaining ideal temperature conditions for individual steps that are repeated cyclically in the presence of standard and sequence-specific chemicals, namely miRNA-specific stem-loop RT primers, miRNA-specific forward and reverse PCR primers, and miRNA-specific MGB probes. In each step, a fluorescence signal released from the probe is measured and the cycle is usually repeated 40-45 times in total. After finishing the program, Cₜ values are read in the individual channels and the results correspond to the original number of monitored nucleic acid molecules. RNA extraction from leukocyte lysate and RT-qPCR analysis are standards in current molecular diagnostic practice. The procedures are fast, simple to perform, and allow for easy automation. Therefore, this diagnostic method is suitable for implementation in most genetic laboratories and for wide use in clinical practice.

The normal distribution of expression of selected miRNAs was determined based on a sample of women who had a physiological pregnancy without complications. These levels were subsequently compared with miRNA levels in samples obtained from women who developed complications associated with a high risk of pregnancy loss during pregnancy, and the results were statistically processed using the Kruskal-Wallis test and the Mann-Whitney test. For each individual pregnancy complication, a specific set of miRNAs was selected. These miRNAs are all dysregulated, i.e., some are up-regulated and some are down-regulated, before the onset of the given complication and their combination enables a prediction of the complication with a sufficiently high sensitivity at a sufficiently low rate of false positives. A generally accepted value is 10% false positive rate.

As can be seen from results presented below, the method makes it possible to effectively predict complications associated with a high risk of pregnancy loss based solely on the results of analysis of a miRNA profile in peripheral venous blood, i.e., without the need for additional clinical examination of women or knowledge of their medical history. Thus, the method is highly objective without the possibility of its distortion by false or erroneous data, and at the same time enables the testing of samples on a large scale, including their evaluation, while preserving the anonymity of the patients. The key aspect is that the method enables prediction of these complications even in first-time mothers or, in general, in women without a previous history of fetal or child death. This is significant from the point of view of clinical practice, as prediction methods for this type of complications are currently not available and increased medical supervision of high-risk patients is possible only on the basis of their previous history of pregnancy loss, i.e., to a very limited extent.

### Prediction of Miscarriage

In women whose pregnancy ended in a spontaneous miscarriage, monitoring the dysregulation of the following 8 miRNAs in whole peripheral venous blood collected during the first trimester proved to be the most suitable for prediction: miR-1-3p (up-regulation), miR-16-5p (up-regulation), miR-17-5p (up-regulation), miR-26a-5p (up-regulation), miR-130b-3p (down-regulation), miR-146a-5p (up-regulation), miR-181a-5p (up-regulation), miR-195-5p (down-regulation). Using this method, without any additional clinical examination of the women or knowledge of their medical history, it was possible to predict 80.52% of cases at 10% false positive rate (FPR).

### Prediction of Stillbirth

In women who gave birth to a dead child, monitoring the dysregulation of the following 11 miRNAs in whole peripheral venous blood collected during the first trimester proved to be the most suitable for prediction: miR-1-3p (up-regulation), miR-16-5p (up-regulation), miR-17-5p (up-regulation), miR-20a-5p (up-regulation), miR-130b-3p (down-regulation), miR-145-5p (down-regulation), miR-146a-5p (up-regulation), miR-181 a-5p (up-regulation), miR-210-3p (down-regulation), miR-342-3p (down-regulation), miR-574-3p (down-regulation). Using this method, without any additional clinical examination of the women or knowledge of their medical history, it was possible to predict 95.8% of cases at 10% FPR. By monitoring the up-regulation of 2 specific miRNAs from this subset (miR-1-3p and miR-181 a-5p), without any additional clinical examination of the women or knowledge of their medical history, it was possible to predict 91.67% of cases at 10% FPR.

### Aggregated, Non-Specific Prediction of Miscarriage and Stillbirth

In women whose pregnancy ended in a spontaneous miscarriage or who gave birth to a dead child, monitoring the dysregulation of the following 9 miRNAs in whole peripheral venous blood collected during the first trimester proved to be the most suitable for prediction: miR-1-3p (up-regulation), miR-16-5p (up-regulation), miR-17-5p (up-regulation), miR-26a-5p (up-regulation), miR-130b-3p (down-regulation), miR-146a-5p (up-regulation), miR-181 a-5p (up-regulation), miR-342-3p (down-regulation), miR-574-3p (down-regulation). Using this method, without any additional clinical examination of the women or knowledge of their medical history, it was possible to predict 99.01% of cases at 10% FPR.

### Prediction of HELLP Syndrome

In women who developed HELLP syndrome during pregnancy, monitoring the up-regulation of the following 6 miRNAs in whole peripheral venous blood collected during the first trimester proved to be the most suitable for prediction: miR-1-3p, miR-17-5p, miR-143-3p, miR-146a-5p, miR-181a-5p, miR-499a-5p. Using this method, without any additional clinical examination of the women or knowledge of their medical history, it was possible to predict 78.6% of cases at 10% FPR.

### BRIEF DESCRIPTION OF DRAWINGS

FIG. 1 depicts ROC (Receiver Operating Characteristic) curves obtained from the statistical analysis of the dysregulation of 8 selected miRNAs (miR-1-3p, miR-16-5p, miR-17-5p, miR-26a-5p, miR-130b-3p, miR-146a-5p, miR-181 a-5p, miR-195-5p) for prediction of miscarriage, both for combined screening and for individual miRNAs.

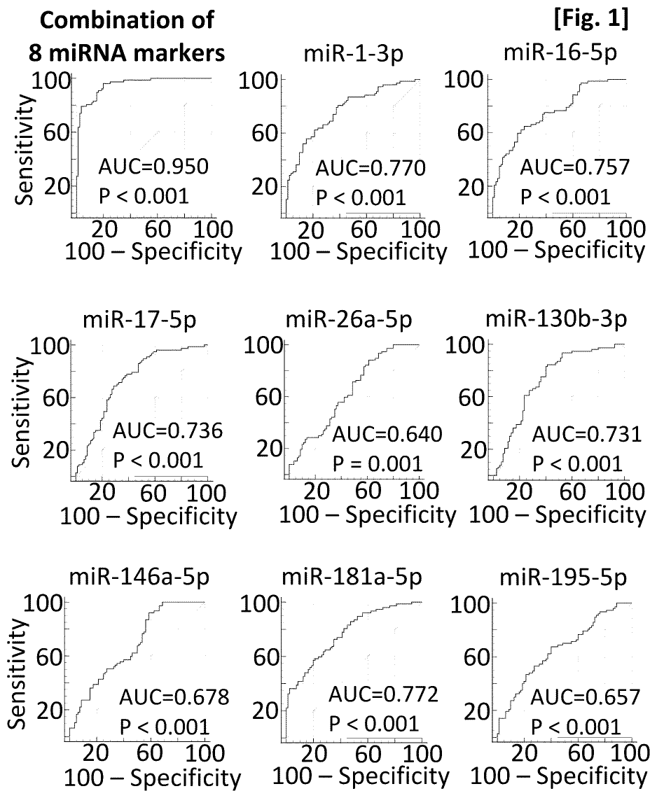

FIG. 2 depicts ROC curves obtained from the statistical analysis of the dysregulation of 11 selected miRNAs (miR-1-3p, miR-16-5p, miR-17-5p, miR-20a-5p, miR-130b-3p, miR-145-5p, miR-146a-5p, miR-181 a-5p, miR-210-3p, miR-342-3p, miR-574-3p) for prediction of stillbirth, both for combined screening and for individual miRNAs.

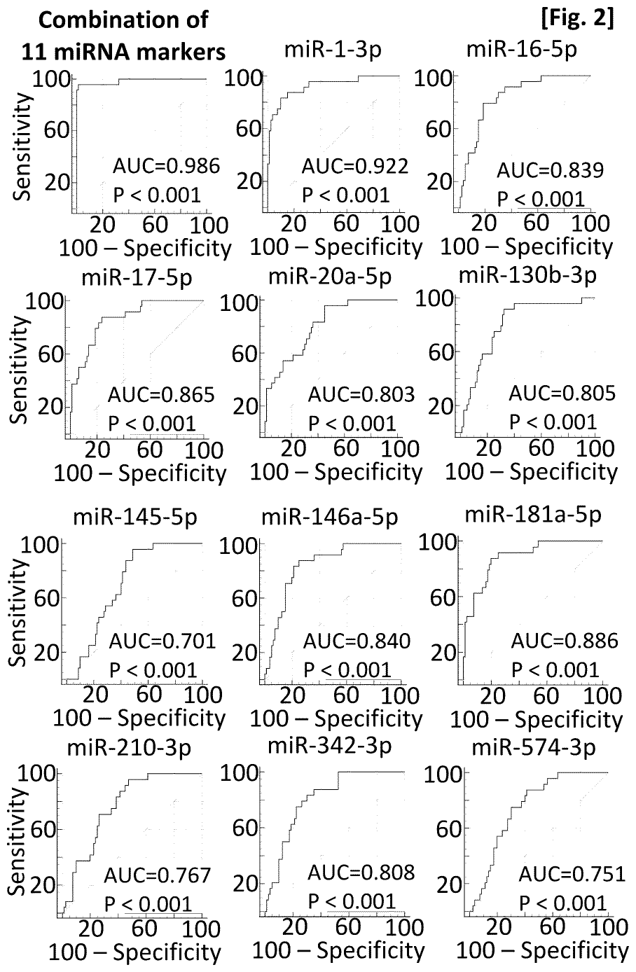

FIG. 3 depicts ROC curve obtained from the statistical analysis of combined screening of the up-regulation of 2 selected miRNAs (miR-1-3p, miR-181 a-5p) for prediction of stillbirth.

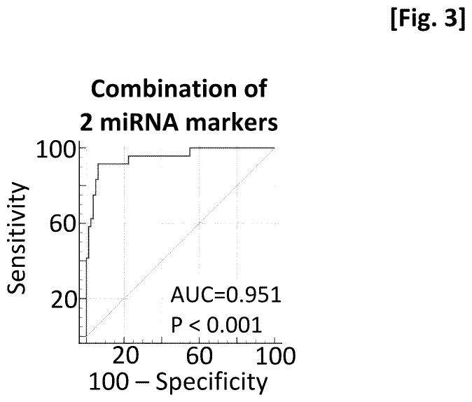

FIG. 4 depicts ROC curves obtained from the statistical analysis of the dysregulation of 9 selected miRNAs (miR-1-3p, miR-16-5p, miR-17-5p, miR-26a-5p, miR-130b-3p, miR-146a-5p, miR-181 a-5p, miR-342-3p, miR-574-3p) for aggregated, non-specific prediction of miscarriage and stillbirth, both for combined screening and for individual miRNAs.

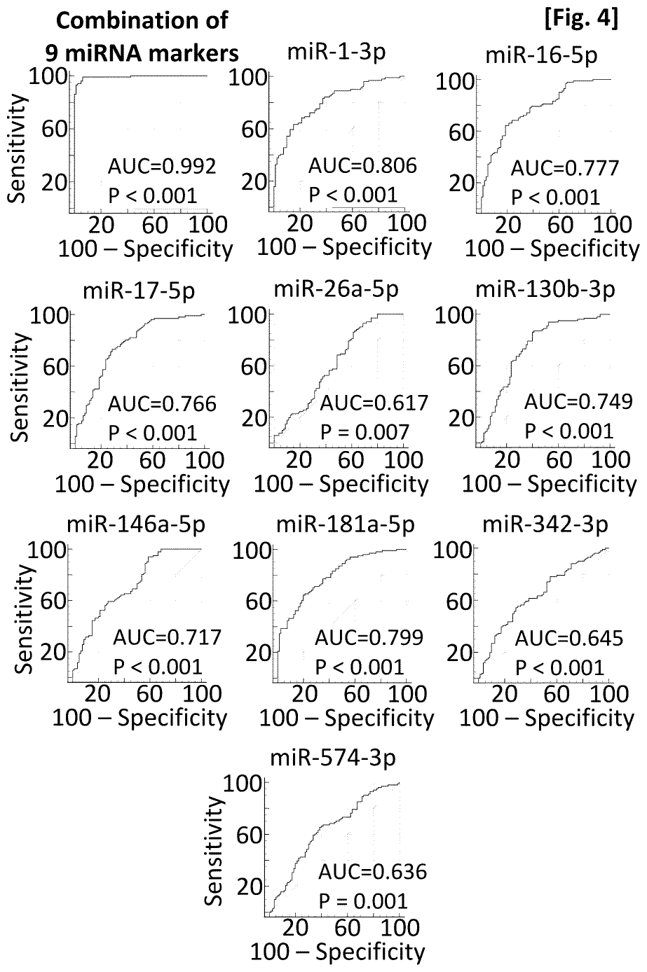

FIG. 5 depicts ROC curves obtained from the statistical analysis of the up-regulation of 6 selected miRNAs (miR-1-3p, miR-17-5p, miR-143-3p, miR-146a-5p, miR-181 a-5p, miR-499a-5p) for prediction of HELLP syndrome, both for combined screening and for individual miRNAs.

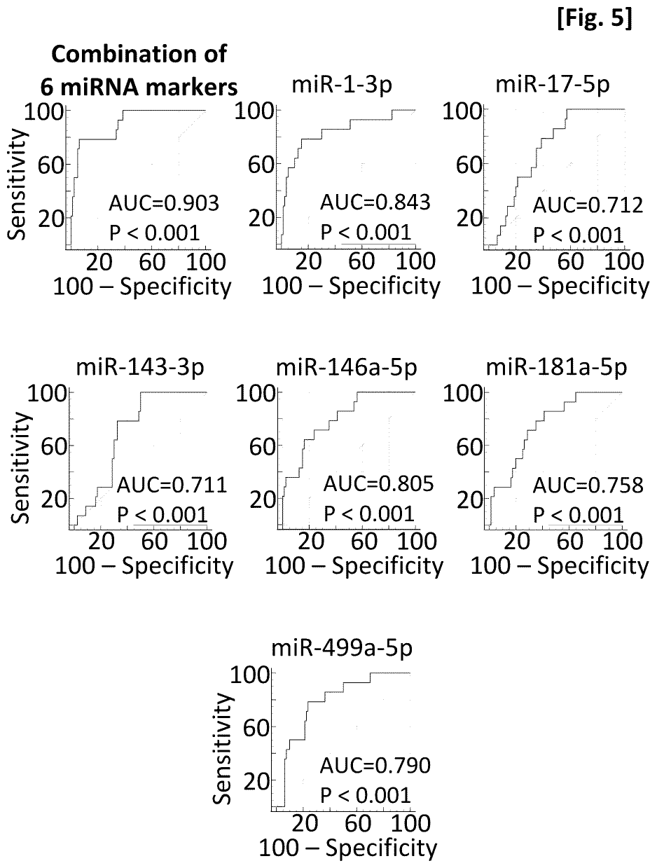

### DESCRIPTION OF EMBODIMENTS

### Example 1

Example 1 describes a general implementation of RT-qPCR analysis determining the amount of miRNA in a tested sample of whole peripheral venous blood and in a reference sample normalized to a simultaneously determined amount of selected endogenous controls (RNU58A a RNU38B).

RNA isolation is performed from thawed leukocyte lysate using a mixture of acidic phenol and chloroform. Long RNAs are further removed from the obtained RNA and short RNAs are concentrated using a column with a glass fiber filter and ethanol of different concentrations in the individual isolation steps. The isolated RNA containing short RNAs is directly used as a template in a two-step RT-qPCR reaction. Reverse transcription takes place under the following conditions: 30 min at 16° C., 30 min at 42° C., and 5 min at 85° C. This is followed by a polymerase chain reaction under the following conditions: 50° C. for 2 min, 95° C. for 10 min, then cycling at 95° C. for 15 s and 60° C. for 1 min. After each cycle, the fluorescence is measured in the FAM and ROX channels (passive reference for fluorescence normalization). The cycling is repeated a total of 40 to 45 times. After the program ends, Cₜ values in the individual channels are read. Within one sample, the Cₜ value in the FAM channel is obtained for the measured miRNA or for the short RNA serving as an endogenous control. These values correspond to the expression of individual genes in the biological sample. The normalized expression value is obtained by subtracting the Cₜ value of the endogenous control (geometric mean of RNU58A and RNU38B) from the Cₜ value of the miRNA in the assayed sample. For relative quantification, the expression of all studied miRNAs and endogenous controls is determined simultaneously also in a reference sample, which is used in all performed analyses.

### Example 2

Example 2 describes a general implementation of statistical analysis of data describing the level of selected miRNAs.

Due to the non-normal distribution of data according to the Shapiro-Wilk test, non-parametric tests are used to evaluate the experimental data. Gene expression of miRNAs is compared between individual groups using the Mann-Whitney test and in case of more than 2 compared groups using the Kruskal-Wallis test followed by a post-hoc analysis. The level of statistical significance is determined on the corrected p-value after applying the Benjamini-Hochberg correction. ROC (Receiver Operating Characteristic) curves are also constructed for the respective miRNAs. The area under the curve, the sensitivity and specificity of individual miRNAs, and the optimal cut-off value (the so-called criterion) are evaluated. Furthermore, the optimal cut-off value and sensitivity of a given miRNA biomarker is determined at 90.0% specificity, which corresponds to information about the percentage of women with increased or decreased expression of a specific miRNA at 10.0% false positive rate (FPR). Furthermore, a combined statistical analysis in the form of logistic regression and ROC analysis is performed in order to select the optimal combination of miRNA biomarkers for the given situation. This application provides the following parameters: area under the curve, sensitivity, specificity, optimal cut-off value, and sensitivity of a given combination of miRNA biomarkers at 90.0% specificity.

### Example 3

Example 3 describes a collection and selection of a suitable set of biological samples for the development of a method for predicting pregnancy complications associated with a high risk of pregnancy loss using cardiovascular miRNAs as biomarkers.

200 μL of whole peripheral venous blood is collected from approximately 12 thousand women in 10th-13th week of pregnancy. A cell lysate of leukocytes is prepared by removing erythrocytes and then stored deep-frozen at −80° C. After the patients give birth, samples from women whose complete state of health during the entire pregnancy is known, including all complications and the course of childbirth, are specifically selected. 80 patients with a physiological course of pregnancy and with a negative result of the first-trimester prenatal screening using an established method utilizing the routine predictive algorithm within a computer application for obstetrics and gynecology databases are selected as a control group. For the study of complications associated with a high risk of pregnancy loss, the following patient groups are selected: 77 patients whose pregnancy ended in a spontaneous miscarriage, 24 patients who gave birth to a dead child, and 14 patients who developed HELLP syndrome during pregnancy. Blood samples of these selected patients are analyzed following the procedure described in Example 1 and levels of selected 15 miRNAs are determined (miR-1-3p, miR-16-5p, miR-17-5p, miR-20a-5p, miR-26a-5p, miR-130b-3p, miR-143-3p, miR-145-5p, miR-146a-5p, miR-181 a-5p, miR-195-5p, miR-210-3p, miR-342-3p, miR-499a-5p, miR-574-3p). For the prediction of individual pregnancy complications with sufficient reliability described in Examples 4-8, more than half of the suitable miRNAs shown in the individual examples are used each time. The highest reliability is achieved by using all suitable miRNAs shown in the individual examples.

### Example 4

Example 4 describes a successful prediction of miscarriage using selected miRNA markers in a selected sample of patients.

77 monitored patients experience spontaneous miscarriage. Blood samples of these selected patients are analyzed following the procedure described in Example 1 and levels of selected 8 miRNAs (miR-1-3p, miR-16-5p, miR-17-5p, miR-26a-5p, miR-130b-3p, miR-146a-5p, miR-181 a-5p, miR-195-5p) are determined. Up-regulation of miR-1-3p, miR-16-5p, miR-17-5p, miR-26a-5p, miR-146a-5p, and miR-181a-5p, whose levels exceed the minimum values determined by a statistical analysis for a 10% FPR, and down-regulation of miR-130b-3p and miR-195-5p, whose levels are below the maximum values determined by a statistical analysis for a 10% FPR, is observed in 62 of 77 patients, which corresponds to a successful prediction in 80.52% of cases. Statistical analysis of the obtained data provides the following values of specificity, sensitivity, 95% Cl, and criterion:

Combined screening of miscarriage (8 selected miRNA)

| Area Under the ROC | 0.950 | Criterion | >0.36685261 |
|---|---|---|---|
| Curve (AUC) | | | |
| Standard Error | 0.0159 | Sensitivity | 96.10 |
| 95% Confidence Interval | 0.903-0.978 | 95% CI | 89.0-99.2 |
| Significance Level P | <0.0001 | Specificity | 80.00 |
| (Area = 0.5) | | | |
| Estimated sensitivity at fixed specificity | | 95% CI | 69.6-88.1 |
| Specificity | 90.00 | +LR | 4.81 |
| Sensitivity | 80.52 | 95% CI | 3.09-7.47 |
| 95% Confidence Interval | 67.53-89.61 | −LR | 0.049 |
| Criterion | >0.550388865 | 95% CI | 0.016-0.15 |

CI = Confidence Interval
LR = Likelihood Ratio

miR-1-3p

| Area Under the ROC Curve (AUC) | 0.770 | Criterion | >0.115119 |
|---|---|---|---|
| Standard Error | 0.0373 | Sensitivity | 80.52 |
| 95% Confidence Interval | 0.696-0.833 | 95% CI | 69.9-88.7 |
| Significance Level P (Area = 0.5) | <0.0001 | Specificity | 61.25 |
| Estimated sensitivity at fixed specificity | | 95% CI | 49.7-71.9 |
| Specificity | 90.00 | +LR | 2.08 |
| Sensitivity | 45.45 | 95% CI | 1.54-2.80 |
| 95% Confidence Interval | 25.97-63.64 | −LR | 0.32 |
| Criterion | >0.377841 | 95% CI | 0.20-0.52 |

CI = Confidence Interval
LR = Likelihood Ratio

miR-16-5p

| Area Under the ROC Curve (AUC) | 0.757 | Criterion | >1.983489 |
|---|---|---|---|
| Standard Error | 0.0378 | Sensitivity | 64.94 |
| 95% Confidence Interval | 0.683-0.822 | 95% CI | 53.2-75.5 |
| Significance Level P (Area = 0.5) | <0.0001 | Specificity | 76.25 |
| Estimated sensitivity at fixed specificity | | 95% CI | 65.4-85.1 |
| Specificity | 90.00 | +LR | 2.73 |
| Sensitivity | 44.16 | 95% CI | 1.79-4.18 |
| 95% Confidence Interval | 28.57-59.74 | −LR | 0.46 |
| Criterion | >3.02212 | 95% CI | 0.33-0.64 |

CI = Confidence Interval
LR = Likelihood Ratio

miR-17-5p

| Area Under the ROC Curve (AUC) | 0.736 | Criterion | >1.780378 |
|---|---|---|---|
| Standard Error | 0.0403 | Sensitivity | 68.83 |
| 95% Confidence Interval | 0.660-0.803 | 95% CI | 57.3-78.9 |
| Significance Level P (Area = 0.5) | <0.0001 | Specificity | 71.25 |
| Estimated sensitivity at fixed specificity | | 95% CI | 60.0-80.8 |
| Specificity | 90.00 | +LR | 2.39 |
| Sensitivity | 24.68 | 95% CI | 1.64-3.49 |
| 95% Confidence Interval | 6.49-41.56 | −LR | 0.44 |
| Criterion | >3.458809 | 95% CI | 0.31-0.63 |

CI = Confidence Interval
LR = Likelihood Ratio

miR-26a-5p

| Area Under the ROC Curve (AUC) | 0.640 | Criterion | >0.525138 |
|---|---|---|---|
| Standard Error | 0.0441 | Sensitivity | 88.31 |
| 95% Confidence Interval | 0.560-0.715 | 95% CI | 79.0-94.5 |
| Significance Level P (Area = 0.5) | 0.0015 | Specificity | 38.75 |
| Estimated sensitivity at fixed specificity | | 95% CI | 28.1-50.3 |
| Specificity | 90.00 | +LR | 1.44 |
| Sensitivity | 23.38 | 95% CI | 1.19-1.75 |
| 95% Confidence Interval | 9.09-37.66 | −LR | 0.30 |
| Criterion | >1.350228 | 95% CI | 0.15-0.59 |

CI = Confidence Interval
LR = Likelihood Ratio

miR-130b-3p

| Area Under the ROC | 0.731 | Criterion | ≤0.604588314 |
|---|---|---|---|
| Curve (AUC) | | | |
| Standard Error | 0.0409 | Sensitivity | 84.42 |
| 95% Confidence Interval | 0.654-0.798 | 95% CI | 74.4-91.7 |
| Significance Level P | <0.0001 | Specificity | 58.75 |
| (Area = 0.5) | | | |
| Estimated sensitivity at fixed specificity | | 95% CI | 47.2-69.6 |
| Specificity | 90.00 | +LR | 2.05 |
| Sensitivity | 20.78 | 95% CI | 1.55-2.70 |
| 95% Confidence Interval | 3.90-40.26 | −LR | 0.27 |
| Criterion | ≤0.235772 | 95% CI | 0.15-0.46 |

CI = Confidence Interval
LR = Likelihood Ratio

miR-146a-5p

| Area Under the ROC | 0.678 | Criterion | >0.879161 |
|---|---|---|---|
| Curve (AUC) | | | |
| Standard Error | 0.0425 | Sensitivity | 92.21 |
| 95% Confidence Interval | 0.599-0.751 | 95% CI | 83.8-97.1 |
| Significance Level P | <0.0001 | Specificity | 41.25 |
| (Area = 0.5) | | | |
| Estimated sensitivity at fixed specificity | | 95% CI | 30.4-52.8 |
| Specificity | 90.00 | +LR | 1.57 |
| Sensitivity | 27.27 | 95% CI | 1.29-1.91 |
| 95% Confidence Interval | 12.99-45.45 | −LR | 0.19 |
| Criterion | >2.744534 | 95% CI | 0.084-0.43 |

CI = Confidence Interval
LR = Likelihood Ratio

miR-181a-5p

| Area Under the ROC Curve (AUC) | 0.772 | Criterion | >0.183648 |
|---|---|---|---|
| Standard Error | 0.0365 | Sensitivity | 85.71 |
| 95% Confidence Interval | 0.699-0.835 | 95% CI | 75.9-92.6 |
| Significance Level P (Area = 0.5) | <0.0001 | Specificity | 52.50 |
| Estimated sensitivity at fixed specificity | | 95% CI | 41.0-63.8 |
| Specificity | 90.00 | +LR | 1.80 |
| Sensitivity | 41.56 | 95% CI | 1.41-2.31 |
| 95% Confidence Interval | 28.57-55.84 | −LR | 0.27 |
| Criterion | >0.521629 | 95% CI | 0.15-0.49 |

CI = Confidence Interval
LR = Likelihood Ratio

miR-195-5p

| Area Under the ROC | 0.657 | Criterion | ≤0.082537076 |
|---|---|---|---|
| Curve (AUC) | | | |
| Standard Error | 0.0434 | Sensitivity | 67.53 |
| 95% Confidence Interval | 0.577-0.731 | 95% CI | 55.9-77.8 |
| Significance Level P | 0.0003 | Specificity | 60.00 |
| (Area = 0.5) | | | |
| Estimated sensitivity at fixed specificity | | 95% CI | 48.4-70.8 |
| Specificity | 90.00 | +LR | 1.69 |
| Sensitivity | 27.27 | 95% CI | 1.24-2.30 |
| 95% Confidence Interval | 12.99-45.63 | −LR | 0.54 |
| Criterion | ≤0.022487627 | 95% CI | 0.37-0.78 |

CI = Confidence Interval
LR = Likelihood Ratio

### Example 5

Example 5 describes a successful prediction of stillbirth using selected miRNA markers in a selected sample of patients.

24 monitored patients give birth to a dead child. Blood samples of these selected patients are analyzed following the procedure described in Example 1 and levels of selected 11 miRNAs (miR-1-3p, miR-16-5p, miR-17-5p, miR-20a-5p, miR-130b-3p, miR-145-5p, miR-146a-5p, miR-181 a-5p, miR-210-3p, miR-342-3p, miR-574-3p) are determined. Up-regulation of miR-1-3p, miR-16-5p, miR-17-5p, miR-20a-5p, miR-146a-5p, and miR-181a-5p, whose levels exceed the minimum values determined by a statistical analysis for a 10% FPR, and down-regulation of miR-130b-3p, miR-145-5p, miR-210-3p, miR-342-3p, and miR-574-3p, whose levels are below the maximum values determined by a statistical analysis for a 10% FPR, is observed in 23 of 24 patients, which corresponds to a successful prediction in 95.8% of cases. Statistical analysis of the obtained data provides the following values of specificity, sensitivity, 95% Cl, and criterion:

Combined screening of stillbirth (11 selected miRNA)

| Area Under the ROC | 0.986 | Criterion | >0.2839140 |
|---|---|---|---|
| Curve (AUC) | | | |
| Standard Error | 0.0137 | Sensitivity | 95.83 |
| 95% Confidence Interval | 0.940-0.999 | 95% CI | 78.9-99.9 |
| Significance Level P | <0.0001 | Specificity | 98.75 |
| (Area = 0.5) | | | |
| Estimated sensitivity at fixed specificity | | 95% CI | 93.2-100.0 |
| Specificity | 90.00 | +LR | 76.67 |
| Sensitivity | 95.83 | 95% CI | 10.91-583.60 |
| 95% Confidence Interval | 79.63-100.00 | −LR | 0.042 |
| Criterion | >0.035796626 | 95% CI | 0.0062-0.29 |

CI = Confidence Interval
LR = Likelihood Ratio

miR-1-3p

| Area Under the ROC | 0.922 | Criterion | >0.377841 |
|---|---|---|---|
| Curve (AUC) | | | |
| Standard Error | 0.0340 | Sensitivity | 83.33 |
| 95% Confidence Interval | 0.853-0.966 | 95% CI | 62.6-95.3 |
| Significance Level P | <0.0001 | Specificity | 90.00 |
| (Area = 0.5) | | | |
| Estimated sensitivity at fixed specificity | | 95% CI | 81.2-95.6 |
| Specificity | 90.00 | +LR | 8.33 |
| Sensitivity | 83.33 | 95% CI | 4.22-16.47 |
| 95% Confidence Interval | 62.50-97.39 | −LR | 0.19 |
| Criterion | >0.377841 | 95% CI | 0.075-0.45 |

CI = Confidence Interval
LR = Likelihood Ratio

miR-16-5p

| Area Under the ROC Curve (AUC) | 0.839 | Criterion | >2.212571 |
|---|---|---|---|
| Standard Error | 0.0415 | Sensitivity | 79.17 |
| 95% Confidence Interval | 0.754-0.903 | 95% CI | 57.8-92.9 |
| Significance Level P (Area = 0.5) | <0.0001 | Specificity | 81.25 |
| Estimated sensitivity at fixed specificity | | 95% CI | 71.0-89.1 |
| Specificity | 90.00 | +LR | 4.22 |
| Sensitivity | 41.67 | 95% CI | 2.56-6.96 |
| 95% Confidence Interval | 16.67-75.00 | −LR | 0.26 |
| Criterion | >3.02212 | 95% CI | 0.12-0.56 |

CI = Confidence Interval
LR = Likelihood Ratio

miR-17-5p

| Area Under the ROC | 0.865 | Criterion | >1.978627 |
|---|---|---|---|
| Curve (AUC) | | | |
| Standard Error | 0.0391 | Sensitivity | 87.50 |
| 95% Confidence Interval | 0.784-0.924 | 95% CI | 67.6-97.3 |
| Significance Level P | <0.0001 | Specificity | 76.25 |
| (Area = 0.5) | | | |
| Estimated sensitivity at fixed specificity | | 95% CI | 65.4-85.1 |
| Specificity | 90.00 | +LR | 3.68 |
| Sensitivity | 50.00 | 95% CI | 2.42-5.61 |
| 95% Confidence Interval | 20.83-70.83 | −LR | 0.16 |
| Criterion | >3.458809 | 95% CI | 0.056-0.48 |

CI = Confidence Interval
LR = Likelihood Ratio

miR-20a-5p

| Area Under the ROC | 0.803 | Criterion | >1.6574 |
|---|---|---|---|
| Curve (AUC) | | | |
| Standard Error | 0.0467 | Sensitivity | 95.83 |
| 95% Confidence Interval | 0.714-0.875 | 95% CI | 78.9-99.9 |
| Significance Level P | <0.0001 | Specificity | 55.00 |
| (Area = 0.5) | | | |
| Estimated sensitivity at fixed specificity | | 95% CI | 43.5-66.2 |
| Specificity | 90.00 | +LR | 2.13 |
| Sensitivity | 41.67 | 95% CI | 1.65-2.75 |
| 95% Confidence Interval | 20.83-62.50 | −LR | 0.076 |
| Criterion | >3.587078 | 95% CI | 0.011-0.52 |

CI = Confidence Interval
LR = Likelihood Ratio

miR-130b-3p

| Area Under the ROC | 0.805 | Criterion | ≤0.44464638 |
|---|---|---|---|
| Curve (AUC) | | | |
| Standard Error | 0.0484 | Sensitivity | 91.67 |
| 95% Confidence Interval | 0.716-0.876 | 95% CI | 73.0-99.0 |
| Significance Level P | <0.0001 | Specificity | 67.50 |
| (Area = 0.5) | | | |
| Estimated sensitivity at fixed specificity | | 95% CI | 56.1-77.6 |
| Specificity | 90.00 | +LR | 2.82 |
| Sensitivity | 33.33 | 95% CI | 2.01-3.95 |
| 95% Confidence Interval | 8.33-62.50 | −LR | 0.12 |
| Criterion | ≤0.235772 | 95% CI | 0.032-0.47 |

CI = Confidence Interval
LR = Likelihood Ratio

miR-145-5p

| Area Under the ROC | 0.701 | Criterion | ≤0.15890505 |
|---|---|---|---|
| Curve (AUC) | | | |
| Standard Error | 0.0504 | Sensitivity | 95.83 |
| 95% Confidence Interval | 0.603-0.786 | 95% CI | 78.9-99.9 |
| Significance Level P | 0.0001 | Specificity | 51.25 |
| (Area = 0.5) | | | |
| Estimated sensitivity at fixed specificity | | 95% CI | 39.8-62.6 |
| Specificity | 90.00 | +LR | 1.97 |
| Sensitivity | 16.67 | 95% CI | 1.55-2.50 |
| 95% Confidence Interval | 0.00-41.67 | −LR | 0.081 |
| Criterion | ≤0.051862838 | 95% CI | 0.012-0.56 |

CI = Confidence Interval
LR = Likelihood Ratio

miR-146a-5p

| Area Under the ROC | 0.840 | Criterion | >1.732512 |
|---|---|---|---|
| Curve (AUC) | | | |
| Standard Error | 0.0416 | Sensitivity | 87.50 |
| 95% Confidence Interval | 0.755-0.904 | 95% CI | 67.6-97.3 |
| Significance Level P | <0.0001 | Specificity | 75.00 |
| (Area = 0.5) | | | |
| Estimated sensitivity at fixed specificity | | 95% CI | 64.1-84.0 |
| Specificity | 90.00 | +LR | 3.50 |
| Sensitivity | 45.83 | 95% CI | 2.33-5.27 |
| 95% Confidence Interval | 16.67-83.33 | −LR | 0.17 |
| Criterion | >2.744534 | 95% CI | 0.057-0.48 |

CI = Confidence Interval
LR = Likelihood Ratio

miR-181a-5p

| Area Under the ROC | 0.886 | Criterion | >0.368016 |
|---|---|---|---|
| Curve (AUC) | | | |
| Standard Error | 0.0359 | Sensitivity | 87.50 |
| 95% Confidence Interval | 0.809-0.940 | 95% CI | 67.6-97.3 |
| Significance Level P | <0.0001 | Specificity | 80.00 |
| (Area = 0.5) | | | |
| Estimated sensitivity at fixed specificity | | 95% CI | 69.6-88.1 |
| Specificity | 90.00 | +LR | 4.38 |
| Sensitivity | 62.50 | 95% CI | 2.75-6.96 |
| 95% Confidence Interval | 37.50-83.33 | −LR | 0.16 |
| Criterion | >0.521629 | 95% CI | 0.054-0.45 |

CI = Confidence Interval
LR = Likelihood Ratio

miR-210-3p

| Area Under the ROC | 0.767 | Criterion | ≤0.1243627 |
|---|---|---|---|
| Curve (AUC) | | | |
| Standard Error | 0.0472 | Sensitivity | 95.83 |
| 95% Confidence Interval | 0.674-0.844 | 95% CI | 78.9-99.9 |
| Significance Level P | <0.0001 | Specificity | 52.50 |
| (Area = 0.5) | | | |
| Estimated sensitivity at fixed specificity | | 95% CI | 41.0-63.8 |
| Specificity | 90.00 | +LR | 2.02 |
| Sensitivity | 37.50 | 95% CI | 1.58-2.58 |
| 95% Confidence Interval | 12.50-66.67 | −LR | 0.079 |
| Criterion | ≤0.0418995 | 95% CI | 0.012-0.55 |

CI = Confidence Interval
LR = Likelihood Ratio

miR-342-3p

| Area Under the ROC | 0.808 | Criterion | ≤1.81094546 |
|---|---|---|---|
| Curve (AUC) | | | |
| Standard Error | 0.0441 | Sensitivity | 83.33 |
| 95% Confidence Interval | 0.719-0.879 | 95% CI | 62.6-95.3 |
| Significance Level P | <0.0001 | Specificity | 70.00 |
| (Area = 0.5) | | | |
| Estimated sensitivity at fixed specificity | | 95% CI | 58.7-79.7 |
| Specificity | 90.00 | +LR | 2.78 |
| Sensitivity | 37.50 | 95% CI | 1.90-4.06 |
| 95% Confidence Interval | 12.50-75.00 | −LR | 0.24 |
| Criterion | ≤0.909153316 | 95% CI | 0.096-0.59 |

CI = Confidence Interval
LR = Likelihood Ratio

miR-574-3p

| Area Under the ROC | 0.751 | Criterion | ≤0.16346195 |
|---|---|---|---|
| Curve (AUC) | | | |
| Standard Error | 0.0488 | Sensitivity | 87.50 |
| 95% Confidence Interval | 0.656-0.830 | 95% CI | 67.6-97.3 |
| Significance Level P | <0.0001 | Specificity | 58.75 |
| (Area = 0.5) | | | |
| Estimated sensitivity at fixed specificity | | 95% CI | 47.2-69.6 |
| Specificity | 90.00 | +LR | 2.12 |
| Sensitivity | 16.67 | 95% CI | 1.57-2.87 |
| 95% Confidence Interval | 0.00-50.00 | −LR | 0.21 |
| Criterion | ≤0.06522 | 95% CI | 0.073-0.62 |

CI = Confidence Interval
LR = Likelihood Ratio

### Example 6

Example 6 describes a successful prediction of stillbirth using selected miRNA markers in a selected sample of patients.

24 monitored patients give birth to a dead child. Blood samples of these selected patients are analyzed following the procedure described in Example 1 and levels of selected 2 miRNAs (miR-1-3p, miR-181 a-5p) are determined. Up-regulation of these selected miRNA biomarkers, whose levels exceed the minimum values determined by a statistical analysis for a 10% FPR, is observed in 22 of 24 patients, which corresponds to a successful prediction in 91.67% of cases. Statistical analysis of the obtained data provides the following values of specificity, sensitivity, 95% Cl, and criterion:

Combined screening of stillbirth (2 selected miRNA)

| Area Under the ROC | 0.951 | Criterion | >0.238192 |
|---|---|---|---|
| Curve (AUC) | | | |
| Standard Error | 0.0260 | Sensitivity | 91.67 |
| 95% Confidence Interval | 0.889-0.983 | 95% CI | 73.0-99.0 |
| Significance Level P | <0.0001 | Specificity | 93.75 |
| (Area = 0.5) | | | |
| Estimated sensitivity at fixed specificity | | 95% CI | 86.0-97.9 |
| Specificity | 90.00 | +LR | 14.67 |
| Sensitivity | 91.67 | 95% CI | 6.22-34.56 |
| 95% Confidence Interval | 62.50-100.00 | −LR | 0.089 |
| Criterion | >0.218347 | 95% CI | 0.024-0.34 |

CI = Confidence Interval
LR = Likelihood Ratio

### Example 7

Example 7 describes a successful aggregated, non-specific prediction of miscarriage and stillbirth using selected miRNA markers in a selected sample of patients.

101 monitored patients experience spontaneous miscarriage or give birth to a dead child. Blood samples of these selected patients are analyzed following the procedure described in Example 1 and levels of selected 9 miRNAs (miR-1-3p, miR-16-5p, miR-17-5p, miR-26a-5p, miR-130b-3p, miR-146a-5p, miR-181 a-5p, miR-342-3p, miR-574-3p) are determined. Up-regulation of miR-1-3p, miR-16-5p, miR-17-5p, miR-26a-5p, miR-146a-5p, and miR-181a-5p, whose levels exceed the minimum values determined by a statistical analysis for a 10% FPR, and down-regulation of miR-130b-3p, miR-342-3p, and miR-574-3p, whose levels are below the maximum values determined by a statistical analysis for a 10% FPR, is observed in 100 of 101 patients, which corresponds to a successful prediction in 99.01% of cases. Statistical analysis of the obtained data provides the following values of specificity, sensitivity, 95% Cl, and criterion:

Combined screening of miscarriage
and stillbirth (9 selected miRNA)

| Area Under the ROC | 0.992 | Criterion | >0.326199 |
|---|---|---|---|
| Curve (AUC) | | | |
| Standard Error | 0.00488 | Sensitivity | 99.01 |
| 95% Confidence Interval | 0.965-0.999 | 95% CI | 94.6-100.0 |
| Significance Level P | <0.0001 | Specificity | 93.75 |
| (Area = 0.5) | | | |
| Estimated sensitivity at fixed specificity | | 95% CI | 86.0-97.9 |
| Specificity | 90.00 | +LR | 15.84 |
| Sensitivity | 99.01 | 95% CI | 6.78-37.02 |
| 95% Confidence Interval | 89.23-100.00 | −LR | 0.011 |
| Criterion | >0.154316302 | 95% CI | 0.0015-0.074 |

CI = Confidence Interval
LR = Likelihood Ratio

miR-1-3p

| Area Under the ROC Curve (AUC) | 0.806 | Criterion | >0.23812 |
|---|---|---|---|
| Standard Error | 0.0318 | Sensitivity | 63.37 |
| 95% Confidence Interval | 0.741-0.861 | 95% CI | 53.2-72.7 |
| Significance Level P (Area = 0.5) | <0.0001 | Specificity | 85.00 |
| Estimated sensitivity at fixed specificity | | 95% CI | 75.3-92.0 |
| Specificity | 90.00 | +LR | 4.22 |
| Sensitivity | 54.46 | 95% CI | 2.46-7.27 |
| 95% Confidence Interval | 38.61-72.28 | −LR | 0.43 |
| Criterion | >0.377841 | 95% CI | 0.33-0.57 |

CI = Confidence Interval
LR = Likelihood Ratio

miR-16-5p

| Area Under the ROC Curve (AUC) | 0.777 | Criterion | >2.212571 |
|---|---|---|---|
| Standard Error | 0.0342 | Sensitivity | 64.36 |
| 95% Confidence Interval | 0.709-0.835 | 95% CI | 54.2-73.6 |
| Significance Level P (Area = 0.5) | <0.0001 | Specificity | 81.25 |
| Estimated sensitivity at fixed specificity | | 95% CI | 71.0-89.1 |
| Specificity | 90.00 | +LR | 3.43 |
| Sensitivity | 43.56 | 95% CI | 2.13-5.54 |
| 95% Confidence Interval | 25.74-62.38 | −LR | 0.44 |
| Criterion | >3.02212 | 95% CI | 0.33-0.58 |

CI = Confidence Interval
LR = Likelihood Ratio

miR-17-5p

| Area Under the ROC Curve (AUC) | 0.766 | Criterion | >1.780378 |
|---|---|---|---|
| Standard Error | 0.0362 | Sensitivity | 73.27 |
| 95% Confidence Interval | 0.698-0.826 | 95% CI | 63.5-81.6 |
| Significance Level P (Area = 0.5) | <0.0001 | Specificity | 71.25 |
| Estimated sensitivity at fixed specificity | | 95% CI | 60.0-80.8 |
| Specificity | 90.00 | +LR | 2.55 |
| Sensitivity | 30.69 | 95% CI | 1.77-3.67 |
| 95% Confidence Interval | 13.86-48.51 | −LR | 0.38 |
| Criterion | >3.458809 | 95% CI | 0.26-0.53 |

CI = Confidence Interval
LR = Likelihood Ratio

miR-26a-5p

| Area Under the ROC Curve (AUC) | 0.617 | Criterion | >0.525138 |
|---|---|---|---|
| Standard Error | 0.0432 | Sensitivity | 86.14 |
| 95% Confidence Interval | 0.542-0.688 | 95% CI | 77.8-92.2 |
| Significance Level P (Area = 0.5) | 0.0068 | Specificity | 38.75 |
| Estimated sensitivity at fixed specificity | | 95% CI | 28.1-50.3 |
| Specificity | 90.00 | +LR | 1.41 |
| Sensitivity | 17.82 | 95% CI | 1.16-1.70 |
| 95% Confidence Interval | 6.93-28.76 | −LR | 0.36 |
| Criterion | >1.350228 | 95% CI | 0.20-0.63 |

CI = Confidence Interval
LR = Likelihood Ratio

miR-130b-3p

| Area Under the ROC | 0.749 | Criterion | ≤0.591670738 |
|---|---|---|---|
| Curve (AUC) | | | |
| Standard Error | 0.0383 | Sensitivity | 86.14 |
| 95% Confidence Interval | 0.679-0.810 | 95% CI | 77.8-92.2 |
| Significance Level P | <0.0001 | Specificity | 60.00 |
| (Area = 0.5) | | | |
| Estimated sensitivity at fixed specificity | | 95% CI | 48.4-70.8 |
| Specificity | 90.00 | +LR | 2.15 |
| Sensitivity | 23.76 | 95% CI | 1.63-2.85 |
| 95% Confidence Interval | 6.93-42.35 | −LR | 0.23 |
| Criterion | ≤0.235772 | 95% CI | 0.14-0.39 |

CI = Confidence Interval
LR = Likelihood Ratio

miR-146a-5p

| Area Under the ROC | 0.717 | Criterion | >0.879161 |
|---|---|---|---|
| Curve (AUC) | | | |
| Standard Error | 0.0385 | Sensitivity | 94.06 |
| 95% Confidence Interval | 0.645-0.781 | 95% CI | 87.5-97.8 |
| Significance Level P | <0.0001 | Specificity | 41.25 |
| (Area = 0.5) | | | |
| Estimated sensitivity at fixed specificity | | 95% CI | 30.4-52.8 |
| Specificity | 90.00 | +LR | 1.60 |
| Sensitivity | 31.68 | 95% CI | 1.32-1.94 |
| 95% Confidence Interval | 16.83-52.48 | −LR | 0.14 |
| Criterion | >2.744534 | 95% CI | 0.063-0.33 |

CI = Confidence Interval
LR = Likelihood Ratio

miR-181a-5p

| Area Under the ROC Curve (AUC) | 0.799 | Criterion | >0.368016 |
|---|---|---|---|
| Standard Error | 0.0321 | Sensitivity | 64.36 |
| 95% Confidence Interval | 0.734-0.855 | 95% CI | 54.2-73.6 |
| Significance Level P (Area = 0.5) | <0.0001 | Specificity | 80.00 |
| Estimated sensitivity at fixed specificity | | 95% CI | 69.6-88.1 |
| Specificity | 90.00 | +LR | 3.22 |
| Sensitivity | 46.53 | 95% CI | 2.03-5.11 |
| 95% Confidence Interval | 32.67-61.52 | −LR | 0.45 |
| Criterion | >0.521629 | 95% CI | 0.34-0.59 |

CI = Confidence Interval
LR = Likelihood Ratio

miR-342-3p

| Area Under the ROC | 0.645 | Criterion | ≤1.797163562 |
|---|---|---|---|
| Curve (AUC) | | | |
| Standard Error | 0.0412 | Sensitivity | 54.46 |
| 95% Confidence Interval | 0.571-0.715 | 95% CI | 44.2-64.4 |
| Significance Level P | 0.0004 | Specificity | 71.25 |
| (Area = 0.5) | | | |
| Estimated sensitivity at fixed specificity | | 95% CI | 60.0-80.8 |
| Specificity | 90.00 | +LR | 1.89 |
| Sensitivity | 26.73 | 95% CI | 1.28-2.79 |
| 95% Confidence Interval | 11.84-45.54 | −LR | 0.64 |
| Criterion | ≤0.909153316 | 95% CI | 0.50-0.82 |

CI = Confidence Interval
LR = Likelihood Ratio

miR-574-3p

| Area Under the ROC | 0.636 | Criterion | ≤0.158363743 |
|---|---|---|---|
| Curve (AUC) | | | |
| Standard Error | 0.0419 | Sensitivity | 65.35 |
| 95% Confidence Interval | 0.561-0.706 | 95% CI | 55.2-74.5 |
| Significance Level P | 0.0012 | Specificity | 61.25 |
| (Area = 0.5) | | | |
| Estimated sensitivity at fixed specificity | | 95% CI | 49.7-71.9 |
| Specificity | 90.00 | +LR | 1.69 |
| Sensitivity | 15.84 | 95% CI | 1.24-2.30 |
| 95% Confidence Interval | 6.93-28.71 | −LR | 0.57 |
| Criterion | ≤0.06522 | 95% CI | 0.41-0.78 |

CI = Confidence Interval
LR = Likelihood Ratio

### Example 8

Example 8 describes a successful prediction of HELLP syndrome using selected miRNA markers in a selected sample of patients.

14 monitored patients develop HELLP syndrome during pregnancy. Blood samples of these selected patients are analyzed following the procedure described in Example 1 and levels of selected 6 miRNAs (miR-1-3p, miR-17-5p, miR-143-3p, miR-146a-5p, miR-181 a-5p, miR-499a-5p) are determined. Up-regulation of these selected miRNA biomarkers, whose levels exceed the minimum values determined by a statistical analysis for a 10% FPR, is observed in 11 of 14 patients, which corresponds to a successful prediction in 78.6% of cases. Statistical analysis of the obtained data provides the following values of specificity, sensitivity, 95% Cl, and criterion:

Combined screening of HELLP syndrome (6 selected miRNA)

| Area Under the ROC | 0.903 | Criterion | >0.266206 |
|---|---|---|---|
| Curve (AUC) | | | |
| Standard Error | 0.0420 | Sensitivity | 78.57 |
| 95% Confidence Interval | 0.824-0.945 | 95% CI | 49.2-95.3 |
| Significance Level P | <0.0001 | Specificity | 93.75 |
| (Area = 0.5) | | | |
| Estimated sensitivity at fixed specificity | | 95% CI | 86.0-97.9 |
| Specificity | 90.00 | +LR | 12.57 |
| Sensitivity | 78.57 | 95% CI | 5.15-30.67 |
| 95% Confidence Interval | 42.86-92.86 | −LR | 0.23 |
| Criterion | >0.162280284 | 95% CI | 0.084-0.62 |

CI = Confidence Interval
LR = Likelihood Ratio

miR-1-3p

| Area Under the ROC | 0.843 | Criterion | >0.23812 |
|---|---|---|---|
| Curve (AUC) | | | |
| Standard Error | 0.0666 | Sensitivity | 78.57 |
| 95% Confidence Interval | 0.753-0.910 | 95% CI | 49.2-95.3 |
| Significance Level P | <0.0001 | Specificity | 85.00 |
| (Area = 0.5) | | | |
| Estimated sensitivity at fixed specificity | | 95% CI | 75.3-92.0 |
| Specificity | 90.00 | +LR | 5.24 |
| Sensitivity | 64.29 | 95% CI | 2.91-9.44 |
| 95% Confidence Interval | 32.10-92.86 | −LR | 0.25 |
| Criterion | >0.377841 | 95% CI | 0.092-0.69 |

CI = Confidence Interval
LR = Likelihood Ratio

miR-17-5p

| Area Under the ROC | 0.712 | Criterion | >1.201854 |
|---|---|---|---|
| Curve (AUC) | | | |
| Standard Error | 0.0590 | Sensitivity | 100.00 |
| 95% Confidence Interval | 0.610-0.801 | 95% CI | 76.8-100.0 |
| Significance Level P | 0.0003 | Specificity | 42.50 |
| (Area = 0.5) | | | |
| Estimated sensitivity at fixed specificity | | 95% CI | 31.5-54.1 |
| Specificity | 90.00 | +LR | 1.74 |
| Sensitivity | 14.29 | 95% CI | 1.44-2.10 |
| 95% Confidence Interval | 0.00-57.14 | −LR | 0.00 |
| Criterion | >3.458809 | 95% CI | |

CI = Confidence Interval
LR = Likelihood Ratio

miR-143-3p

| Area Under the ROC | 0.711 | Criterion | >0.03605 |
|---|---|---|---|
| Curve (AUC) | | | |
| Standard Error | 0.0559 | Sensitivity | 100.00 |
| 95% Confidence Interval | 0.608-0.800 | 95% CI | 76.8-100.0 |
| Significance Level P | 0.0002 | Specificity | 50.00 |
| (Area = 0.5) | | | |
| Estimated sensitivity at fixed specificity | | 95% CI | 38.6-61.4 |
| Specificity | 90.00 | +LR | 2.00 |
| Sensitivity | 14.29 | 95% CI | 1.61-2.49 |
| 95% Confidence Interval | 0.00-50.00 | −LR | 0.00 |
| Criterion | >0.129856 | 95% CI | |

CI = Confidence Interval
LR = Likelihood Ratio

miR-146-5p

| Area Under the ROC | 0.805 | Criterion | >2.194722 |
|---|---|---|---|
| Curve (AUC) | | | |
| Standard Error | 0.0589 | Sensitivity | 64.29 |
| 95% Confidence Interval | 0.711-0.880 | 95% CI | 35.1-87.2 |
| Significance Level P (Area = 0.5) | <0.0001 | Specificity | 83.75 |
| Estimated sensitivity at fixed specificity | | 95% CI | 73.8-91.1 |
| Specificity | 90.00 | +LR | 3.96 |
| Sensitivity | 35.71 | 95% CI | 2.10-7.45 |
| 95% Confidence Interval | 14.29-64.29 | −LR | 0.43 |
| Criterion | >2.744534 | 95% CI | 0.21-0.87 |

CI = Confidence Interval
LR = Likelihood Ratio

miR-181a-5p

| Area Under the ROC | 0.758 | Criterion | >0.222962 |
|---|---|---|---|
| Curve (AUC) | | | |
| Standard Error | 0.0620 | Sensitivity | 85.71 |
| 95% Confidence Interval | 0.659-0.840 | 95% CI | 57.2-98.2 |
| Significance Level P | <0.0001 | Specificity | 58.75 |
| (Area = 0.5) | | | |
| Estimated sensitivity at fixed specificity | | 95% CI | 47.2-69.6 |
| Specificity | 90.00 | +LR | 2.08 |
| Sensitivity | 28.57 | 95% CI | 1.48-2.91 |
| 95% Confidence Interval | 7.14-57.14 | −LR | 0.24 |
| Criterion | >0.521629 | 95% CI | 0.067-0.89 |

CI = Confidence Interval
LR = Likelihood Ratio

miR-499a-5p

| Area Under the ROC Curve (AUC) | 0.790 | Criterion | >0.59486 |
|---|---|---|---|
| Standard Error | 0.0599 | Sensitivity | 78.57 |
| 95% Confidence Interval | 0.694-0.867 | 95% CI | 49.2-95.3 |
| Significance Level P (Area = 0.5) | <0.0001 | Specificity | 76.25 |
| Estimated sensitivity at fixed specificity | | 95% CI | 65.4-85.1 |
| Specificity | 90.00 | +LR | 3.31 |
| Sensitivity | 50.00 | 95% CI | 2.05-5.34 |
| 95% Confidence Interval | 14.29-78.57 | −LR | 0.28 |
| Criterion | >1.191424 | 95% CI | 0.10-0.77 |

CI = Confidence Interval
LR = Likelihood Ratio

### INDUSTRIAL APPLICABILITY

Method of prediction of pregnancy complications associated with a high risk of pregnancy loss based on the expression profile of cardiovascular miRNAs is industrially applicable in the clinical practice of gynecology and obstetrics in the laboratory analysis of collected samples of biological material.

1. Method of prediction of pregnancy complications associated with a high risk of pregnancy loss in the form of miscarriage, stillbirth, or HELLP syndrome, regardless of previous affliction, characterized in that pregnant woman is screened to determine the expression profile of two or more miRNAs in whole peripheral venous blood collected between the 10th and the 13th gestational week, whereas said two or more miRNAs are selected from the group miR-1-3p, miR-16-5p, miR-17-5p, miR-20a-5p, miR-26a-5p, miR-130b-3p, miR-143-3p, miR-145-5p, miR-146a-5p, miR-181 a-5p, miR-195-5p, miR-210-3p, miR-342-3p, miR-499a-5p, miR-574-3p.

2. Method according to claim 1, characterized in that the screened miRNAs for prediction of miscarriage are miR-1-3p, miR-16-5p, miR-17-5p, miR-26a-5p, miR-130b-3p, miR-146a-5p, miR-181 a-5p, and miR-195-5p.

3. Method according to claim 1, characterized in that the screened miRNAs for prediction of stillbirth are miR-1-3p, miR-16-5p, miR-17-5p, miR-20a-5p, miR-130b-3p, miR-145-5p, miR-146a-5p, miR-181a-5p, miR-210-3p, miR-342-3p, and miR-574-3p.

4. Method according to claim 1, characterized in that the screened miRNAs for prediction of stillbirth are miR-1-3p and miR-181 a-5p.

5. Method according to claim 1, characterized in that the screened miRNAs for non-specific prediction of miscarriage and stillbirth are miR-1-3p, miR-16-5p, miR-17-5p, miR-26a-5p, miR-130b-3p, miR-146a-5p, miR-181 a-5p, miR-342-3p, and miR-574-3p.

6. Method according to claim 1, characterized in that the screened miRNAs for prediction of HELLP syndrome are miR-1-3p, miR-17-5p, miR-143-3p, miR-146a-5p, miR-181 a-5p, and miR-499a-5p.
